SEQUENCE LISTING (5 sequences)

SEQ ID NO: 1 - DNA, 360 bp, synthetic construct
  source 1..360: mol_type = other DNA
caggtgcagttggtagagagtggtggaggccttgtgcagccaggaggcagccttcgtctgtcctgtgcagcctctggatctgcttcttctgactacgttatggcgtggtttcgccaggcccccggacaaggcttagaggcggtggcggcaatcaactggactggtgacagtacttactacgcagactcagtgaagggacgttttacgatctcacgtgataactcaaagaacactttgtatctgcaaatgaactcacttcgtgctgaggacacggctgtctattactgtgcggcataccgttacatctgggacttcgttccgcattattggggccaaggcacccttgtaacagtttcaagc

SEQ ID NO: 2 - protein, 9 aa, synthetic construct
SASSDYVMA

SEQ ID NO: 3 - protein, 6 aa, synthetic construct
NWTGDS

SEQ ID NO: 4 - protein, 11 aa, synthetic construct
AYRYIWDFVPH

SEQ ID NO: 5 - protein, 120 aa, synthetic construct
QVQLVESGGGLVQPGGSLRLSCAASGSASSDYVMAWFRQAPGQGLEAVAAINWTGDSTYYADSVKGRFTISRDNSKNTLYLQMNSLRAEDTAVYYCAAYRYIWDFVPHYWGQGTLVTVSS

(19) United States
(12) Patent Application Publication
(10) Pub. No.: US 2026/0070971 A1
(43) Pub. Date: Mar. 12, 2026

(54) NANOBODY THAT SPECIFICALLY BIND TO VASPIN, AND COMPOSITIONS COMPRISING SAME FOR PREVENTING OR TREATING OSTEOARTHRITIS

(71) Applicant: Research & Business Foundation SUNGKYUNKWAN UNIVERSITY, Suwon-si, Gyeonggi-do (KR)

(72) Inventors: Siyoung Yang, Pyeongtaek-si, Gyeonggi-do (KR); Jimin Jeon, Suwon-si, Gyeonggi-do (KR); Chanmi Cho, Suwon-si, Gyeonggi-do (KR); Hahyeong Jang, Suwon-si, Gyeonggi-do (KR)

(73) Assignee: Research & Business Foundation SUNGKYUNKWAN UNIVERSITY, Suwon-si, Gyeonggi-do (KR)

(21) Appl. No.: 19/277,413

(22) Filed: Jul. 23, 2025

(30) Foreign Application Priority Data
Sep. 6, 2024 (KR) 10-2024-0121449

(51) Int. Cl. C07K 16/26; A61P 19/02

(52) U.S. Cl. CPC C07K 16/26; A61P 19/02; C07K 2317/24; C07K 2317/569

(57) ABSTRACT

The present invention relates to a nanobody that specifically binds to vaspin (visceral adipose tissue-derived serine protease inhibitor) and a pharmaceutical composition comprising the same for the prevention or treatment of osteoarthritis, wherein the invention is based on the finding that vaspin protein expression is elevated in cartilage tissues and chondrocytes under osteoarthritis-inducing conditions, leading to increased cartilage degradation and a high-affinity nanobody specific for vaspin was screened using bio-panning of a VHH phage display library. The screened nanobody was shown to suppress cartilage destruction in osteoarthritis models.

### CROSS-REFERENCE TO RELATED APPLICATION

This application claims the benefit of Korean Patent Application Nos. 10-2024-0121449 filed on Sep. 6, 2024, in the Korean Intellectual Property Office, the entire disclosure of which is incorporated herein by reference for all purposes.

### SEQUENCE LISTING

This application contains a Sequence Listing submitted via USPTO Patent Center and hereby incorporated by reference in its entirety. The Sequence Listing is named \2280-594.xml, created on Jul. 22, 2025, and 5,985 bytes in size.

### TECHNICAL FIELD

The present invention relates to a nanobody that specifically binds to vaspin and to a pharmaceutical composition comprising the same for the prevention or treatment of osteoarthritis.

### BACKGROUND

Osteoarthritis (OA) is a degenerative joint disease in which progressive pathological changes occur in the articular cartilage and adjacent bone of synovial joints. It is characterized by gradual loss of cartilage, hypertrophy of subchondral bone, osteophyte formation at the joint margins, and nonspecific synovial inflammation. Cartilage degeneration in OA includes an increase in water content, fibrillation due to cartilage surface cracking and tearing, and exposure of the underlying bone. Although chondrocytes attempt to regenerate cartilage, degradation proceeds at a faster rate than repair, ultimately resulting in net cartilage loss. OA progression is accompanied by bone remodeling that causes joint deformities and dysfunction, along with thickening of periarticular soft tissues.

In healthy joints, cartilage serves as a smooth, slippery tissue covering the ends of bones, allowing for frictionless movement and shock absorption. In OA, the surface layer of cartilage becomes eroded, leading to direct bone-on-bone contact. This causes joint pain, swelling, and loss of mobility. OA is age-related and primarily affects weight-bearing joints such as the knees, hips, fingers, and lower spine.

Current treatment approaches are limited to physical therapies aimed at controlling pain and stiffness and maintaining joint function through daily activity. Strengthening muscles and bones or improving flexibility may alleviate symptoms; however, no approved pharmacological therapy exists that fundamentally halts or reverses disease progression.

Recently, nanobodies—single-domain antibodies derived from the variable region of heavy-chain-only antibodies naturally found in camelids (e.g., camels, llamas, alpacas)—have gained attention in therapeutics and diagnostics. Owing to their small size and high structural stability, nanobodies can overcome several limitations of traditional monoclonal antibodies. While conventional antibodies generally have flat or shallow antigen-binding surfaces that are inefficient at binding concave targets such as enzyme active sites, nanobodies have a convex paratope that allows them to access such cryptic epitopes.

Notably, nanobodies possess longer CDR-H3 loops than human antibodies—approximately 19 amino acids on average versus 12 in human antibodies—enabling the formation of pronounced loops and facilitating high-affinity binding. Their structurally simple, compact format enhances stability and production efficiency compared to conventional antibodies.

### RELATED ART

KR 10-2015-0058365 A (Published May 28, 2015)
KR 10-1770252 B1 (Registered Aug. 22, 2017)

### Technical Problem

The present invention aims to identify the critical role of the vaspin protein in osteoarthritis (OA) and to provide a novel therapeutic nanobody that targets vaspin. This nanobody, screened via VHH phage display library biopanning, effectively reduces vaspin activity and exhibits therapeutic efficacy in OA models.

### Technical Solution

A nanobody or antigen-binding fragment thereof that specifically binds to vaspin.

A polynucleotide encoding the nanobody.

A recombinant vector comprising the polynucleotide.

A host cell comprising the recombinant vector.

A method for producing the nanobody or antigen-binding fragment by expressing and purifying the nanobody in said host cell.

A pharmaceutical composition for preventing or treating osteoarthritis, comprising the nanobody or antigen-binding fragment and a pharmaceutically acceptable carrier.

The present invention confirms that vaspin protein expression is upregulated in articular cartilage and chondrocytes under osteoarthritis-inducing conditions, contributing to cartilage destruction. High-affinity nanobodies targeting vaspin were successfully screened using a VHH phage display library. The selected nanobodies significantly inhibited OA progression in animal models by reducing cartilage degradation, suggesting their therapeutic potential for OA treatment.

### DESCRIPTION OF THE DRAWINGS

FIG. 1: Alcian blue and immunohistochemical staining of vaspin protein in human cartilage tissue.

FIG. 2: Safranin-O and immunohistochemical staining of vaspin protein in mouse cartilage tissue.

FIG. 3: Comparison of cartilage damage severity by Safranin-O staining and OARSI score in cartilage-specific vaspin-overexpressing transgenic mice subjected to DMM-induced OA.

FIG. 4: Workflow for biopanning of vaspin-specific nanobodies from a synthetic phage-displayed nanobody (VHH) library.

FIG. 5: Binding affinity and kinetics of screened vaspin-specific nanobodies analyzed by SPR (Surface Plasmon Resonance).

FIG. 6: Protein-protein docking simulation results showing the interaction between the screened nanobody and vaspin.

FIG. 7: Safranin-O staining and OARSI scoring of cartilage tissue from OA model animals.

FIG. 8: Schematic diagram of the experimental procedure for evaluating the therapeutic efficacy of the vaspin-targeting nanobody in an OA animal model.

FIG. 9: Comparison of cartilage damage (Safranin-O staining and OARSI scoring) in OA model animals treated with the vaspin-targeting nanobody.

### DETAILED DESCRIPTION OF THE INVENTION

Unless otherwise defined, all terms used in the present specification are intended to have the meanings commonly understood by those of ordinary skill in the art to which this invention belongs, in consideration of the function of the invention. Common technical and scientific terms are used as consistently as possible with their conventional meanings. In certain cases, terms may have been arbitrarily selected by the Applicant; such terms will be clearly defined in the appropriate sections of the specification.

Accordingly, the terminology used herein should not be interpreted based merely on the literal meaning of the terms, but rather in light of the specification as a whole and the intended scope of the invention. Terms defined in commonly used dictionaries should be interpreted in accordance with their meanings in the context of the relevant technical field, and not in an idealized or overly formal sense unless explicitly defined otherwise in this application.

The present invention provides a nanobody or antigen-binding fragment thereof that specifically binds to vaspin.

As used herein, the term “nanobody” refers to a single-domain antibody analog composed of only one variable region of a heavy chain. It is used interchangeably with “VHH antibody” or “sybody.” A nanobody represents the smallest fully functional antigen-binding fragment. Typically, a nanobody can be generated by isolating antibodies that naturally lack light chains and the first constant domain of the heavy chain (CH1), and cloning the variable region of the heavy chain to produce a single-domain antibody (Variable Domain of Heavy-chain Antibodies, VHH).

Nanobodies can be derived by engineering antibody fragments obtained from camelid species such as camels, llamas, or alpacas. They are approximately one-tenth the size of conventional antibodies and exhibit advantages such as high thermal and chemical stability, ease of administration, and high production yield. Structurally, nanobodies generally comprise four framework regions (FRs) and three complementarity-determining regions (CDRs) in the following order: FR1-CDR1-FR2-CDR2-FR3-CDR3-FR4. In some instances, a nanobody may include only part of FR1 and/or FR4 or may lack one or two FRs, provided that it retains sufficient antigen-binding capacity and specificity.

Nanobodies are also referred to as single-domain antibodies (sdAbs), and these terms are used interchangeably in the context of the present invention.

In the present invention, the term “antigen-binding fragment” refers to a polypeptide that retains the ability to specifically bind to the same antigen as the nanobody or competes with the nanobody for antigen binding-commonly referred to as the “antigen-binding portion.” Such antigen-binding fragments may be obtained by recombinant DNA technology or by enzymatic or chemical cleavage of the nanobody described herein.

In certain embodiments, the antigen-binding fragment may be truncated at the N-terminus and/or C-terminus relative to the full-length nanobody, and may retain only part of framework regions FR1 and/or FR4 or may lack one or two framework regions, provided that the fragment substantially preserves the binding affinity and specificity of the full-length nanobody.

The antigen-binding fragments of nanobodies can be obtained from nanobodies (e.g., those provided in the present invention) by standard techniques well known to those skilled in the art, including recombinant DNA methods or enzymatic or chemical cleavage. These fragments may be screened for binding specificity in the same manner as the full-length nanobodies.

The nanobody or antigen-binding fragment described in the present invention typically comprises three complementarity-determining regions (CDRs). The precise boundaries of the CDRs may be determined using well-established numbering schemes, such as the Kabat numbering system, Chothia system, or IMGT numbering system.

In one embodiment, the nanobody or antigen-binding fragment comprises: a CDR1 having the amino acid sequence shown in SEQ ID NO:2, a CDR2 having the amino acid sequence shown in SEQ ID NO:3, and a CDR3 having the amino acid sequence shown in SEQ ID NO:4.

In a preferred embodiment, the nanobody is a humanized recombinant nanobody.

In the present invention, the humanized recombinant nanobody refers to a genetically engineered non-human antibody in which the amino acid sequence has been modified to increase homology with human antibody sequences. Typically, the CDR regions of a humanized antibody are partially or wholly derived from a non-human donor antibody, whereas the non-CDR regions (e.g., variable regions and/or constant framework regions, FRs) are partially or wholly derived from a human immunoglobulin (acceptor antibody). In certain embodiments, the CDR regions of the humanized antibody are derived from a non-human donor antibody, while the non-CDR regions (such as the variable and/or constant framework regions) are derived from a human immunoglobulin acceptor antibody. The humanized antibody retains the desirable characteristics of the donor antibody, such as antigen specificity, binding affinity, and reactivity, but is not limited thereto. The donor antibody may be, for example, an antibody derived from a camelid species (e.g., camel, llama, or alpaca) having the desired properties. To generate a humanized antibody, the CDR regions from the antibody of the immunized animal may be inserted into human framework sequences using methods well known in the art. With respect to nanobodies, the humanized antibody may be a humanized VHH, wherein one or more framework regions are substantially replaced with corresponding human framework sequences. In some cases, specific framework residues of the human immunoglobulin may be substituted with corresponding non-human residues. Furthermore, the humanized VHH may contain residues not found in the original VHH or human framework sequence, provided such modifications improve or optimize the performance of the VHH or VHH-containing polypeptide.

In the present invention, the framework region (FR residue) refers to amino acid residues located in the variable region of the nanobody or antigen-binding fragment thereof, excluding the complementarity determining region (CDR) residues.

In one aspect of the present invention, the nanobody or antigen-binding fragment that specifically binds to Vaspin is screened via bio-panning using a VHH phage display library. The screened nanobody exhibits high binding affinity to Vaspin, with a kinetic association rate constant (kon) of 1.077×10⁶ M⁻¹·s⁻¹, a dissociation rate constant (koff) of 7.413×10⁻³ s⁻¹, and an equilibrium dissociation constant (KD) of 6.88 nM.

The term “binding affinity” herein refers to the strength of the binding interaction between the nanobody and its target antigen. The affinity of a particular interaction may be represented by the KD (steady-state dissociation constant), which is calculated as the ratio of koff to kon, based on the concentrations and actual rates of association and dissociation.

In a specific embodiment, the nanobody comprises the following amino acid residues at designated positions: arginine (Arg, R) at position 100, tyrosine (Tyr, Y) at position 101, aspartic acid (Asp, D) at position 31, and isoleucine (Ile, I) at position 102.

More specifically, the nanobody comprises the amino acid sequence set forth in SEQ ID NO: 5.

The present invention further provides a polynucleotide encoding the nanobody or the antigen-binding fragment thereof.

In one embodiment, the polynucleotide comprises the nucleotide sequence set forth in SEQ ID NO: 1.

The invention also provides a recombinant vector comprising the aforementioned polynucleotide.

As used herein, the term “vector” refers to a nucleic acid delivery vehicle into which the polynucleotide may be inserted. When the vector allows expression of the polypeptide encoded by the inserted polynucleotide, it is referred to as an expression vector. The vector may be introduced into a host cell via transformation, transfection, or infection, thereby enabling expression of the genetic elements carried by the vector in the host cell.

The invention also provides a host cell comprising the recombinant vector described above.

In another aspect, the present invention provides a method for producing a nanobody or antigen-binding fragment that specifically binds to Vaspin, the method comprising culturing the host cell described above and recovering the nanobody or antigen-binding fragment therefrom.

Furthermore, the present invention provides a pharmaceutical composition for preventing or treating osteoarthritis, comprising the nanobody or antigen-binding fragment thereof.

The pharmaceutical composition of the present invention may be formulated in a unit dosage form or packaged in a multi-dose container using a pharmaceutically acceptable carrier according to standard practices known to those skilled in the art.

The pharmaceutically acceptable carriers included in the pharmaceutical composition of the present invention are those conventionally used in the art and may include, but are not limited to: lactose, dextrose, sucrose, sorbitol, mannitol, starch, acacia gum, calcium phosphate, alginates, gelatin, calcium silicate, microcrystalline cellulose, polyvinylpyrrolidone, cellulose, water, syrup, methylcellulose, methylparaben, propylparaben, talc, magnesium stearate, and mineral oil. In addition to these components, the pharmaceutical composition of the present invention may further comprise lubricants, wetting agents, sweeteners, flavoring agents, emulsifying agents, suspending agents, and preservatives.

The amount of such additives contained in the pharmaceutical composition is not particularly limited and may be appropriately adjusted within the range commonly used in pharmaceutical formulation.

The pharmaceutical composition of the present invention may be formulated into one or more topical dosage forms selected from the group consisting of aqueous solutions, suspensions, emulsions, injections, pills, capsules, granules, tablets, creams, gels, patches, sprays, ointments, liniments, lotions, pastes, and cataplasms.

The pharmaceutical composition of the present invention may further comprise pharmaceutically acceptable carriers and diluents for formulation. Such carriers and diluents may include, but are not limited to: excipients such as starch, sugars, and mannitol; fillers and extenders such as calcium phosphate; cellulose derivatives such as carboxymethylcellulose and hydroxypropylcellulose; binders such as gelatin, alginates, and polyvinylpyrrolidone; lubricants such as talc, calcium stearate, hydrogenated castor oil, and polyethylene glycol; disintegrants such as povidone and crospovidone; and surfactants such as polysorbates, cetyl alcohol, and glycerol. These pharmaceutically acceptable carriers and diluents should be biologically and physiologically compatible with the subject. Examples of diluents include saline, aqueous buffer solutions, solvents, and/or dispersion media, but are not limited thereto.

The pharmaceutical composition of the present invention may be administered orally or non-orally (e.g., intravenously, subcutaneously, intraperitoneally, or topically), depending on the intended method. For oral administration, the composition may be formulated as tablets, troches, lozenges, aqueous suspensions, oily suspensions, powders, granules, emulsions, hard capsules, soft capsules, syrups, or elixirs. For non-oral administration, the composition may be formulated as injectable solutions, suppositories, inhalable powders for respiratory delivery, aerosol sprays, ointments, topical powders, oils, or creams.

The dosage of the pharmaceutical composition may vary depending on the patient's condition, body weight, age, gender, health status, diet, specific characteristics, properties of the formulation, disease severity, dosing time, route of administration, dosing period or interval, rate of excretion, and drug form. Such dosage may be appropriately selected by a person skilled in the art. For example, the dosage may range from about 0.1 to 10,000 mg/kg, and may be administered once or several times daily.

The effective amount and dosage regimen of the pharmaceutical composition may vary depending on the formulation method, administration route, and timing, and can be readily determined and prescribed by those skilled in the art for the intended therapeutic purpose. The pharmaceutical composition may be administered once daily or in divided doses.

The following examples and experimental procedures are provided to aid in the understanding of the invention and to exemplify specific embodiments thereof. However, these are not intended to limit the scope of the invention in any way. The examples are provided to more fully illustrate the invention to those of ordinary skill in the art.

### EXAMPLES

The following Experimental Examples are provided to describe general experimental procedures that are commonly applied to each of the embodiments of the present invention.

### Example 1. Experimental Method

### 1-1. Human Osteoarthritis Cartilage Samples and Mouse OA Model

Human articular cartilage samples were obtained from individuals aged between 63 and 80 years who were undergoing total knee arthroplasty. Written informed consent was obtained from all patients, and all procedures were approved by the Institutional Review Board of the Catholic University of Korea (Approval No. UC14CNSI0150).

Male C57BL/6 mice were housed at 23° C. in cages with a 12-hour light/dark cycle and had free access to food and water. To generate cartilage-specific Vaspin-overexpressing transgenic mice (Col2a1-Vaspin TG), a Col2a1 promoter and enhancer (Macrogen, Korea) were used. Genotyping was performed on Col2a1-Vaspin TG mice.

To establish the osteoarthritis (OA) mouse model, 12-week-old male TG, KO, and WT littermate mice were subjected to destabilization of the medial meniscus (DMM) surgery. After 12 weeks post-surgery, mice were euthanized and tissues were analyzed using histological and biochemical methods. The spontaneous development of OA was also evaluated in 60-week-old Col2a1-Vaspin TG and WT mice.

### 1-2. Histology and Immunohistochemistry

Paraffin-embedded sections of intact and damaged human cartilage were subjected to Alcian blue staining and immunohistochemical (IHC) analysis for Vaspin protein.

Mouse cartilage samples were fixed in paraformaldehyde, decalcified, embedded in paraffin, sectioned at 6 μm thickness, and stained with Safranin-O using standard protocols. For IHC analysis of cartilage sections, antigen retrieval was performed, and the sections were incubated overnight with primary antibodies against Vaspin (bs-7536R; Bioss, USA), Mmp3 (66338-1-Ig; Proteintech, USA), Mmp13 (18165-1-AP; Proteintech, USA), and Cox2 (66351-1-Ig; Proteintech, USA). Detection was performed using the REAL™ EnVision™ Detection System Kit (K5007; Agilent, USA).

Cartilage destruction was evaluated in a blinded manner using the OARSI scoring system, subchondral bone plate (SBP) thickness scoring, and osteophyte formation scoring criteria.

### 1-3. In Vitro Screening of Vas-Nanobody

To screen for Vaspin-specific nanobodies, a humanized synthetic VHH library (hNbBcll10FGLA) was utilized. This phage-displayed synthetic nanobody library contains over 10¹¹ unique clones with diverse lengths and sequences for CDR1, CDR2, and CDR3. CDR1 and CDR2 were fixed at 9 and 6 amino acids, respectively, while CDR3 was diversified from 6 to 25 amino acids to maximize sequence variation.

Bio-panning was conducted to enrich phages displaying nanobodies that specifically bind to Vaspin. In brief, purified Vaspin antigen (100 μg/mL; Y-biologics) was coated onto immunotubes and incubated overnight at 4° C. The tubes were blocked with 5% skim milk (Thermo Fisher Scientific, USA) in DPBS for 1 hour at 37° C., followed by incubation with 1 mL of the nanobody phage library (10¹² PFU/mL) for 2 hours. The tubes were then washed 10 times with DPBS containing 0.05% Tween-20 and the bound phages were eluted using 1 mL of 100 mM triethylamine (Sigma, USA) for 10 minutes. The eluted phages were neutralized with 1 M Tris-HCl (pH 7.5) and used to infect E. coli TG1 (OD600=0.5-0.6) at 37° C. for 30 minutes.

This screening process was repeated three times to increase the stringency of binding (with washing steps increased by 10-fold each round). Individual colonies were randomly selected from the third round of panning, cultured to OD600=0.6, and induced with IPTG at 28° C. for 12 hours. Vaspin-specific nanobodies (termed Vas-nanobody) were selected using indirect ELISA with culture supernatants.

### 1-4. Surface Plasmon Resonance (SPR) Analysis

Binding affinity was determined using a Biacore T200 system (Cytiva, Uppsala, Sweden) equipped with a CM5 sensor chip. Experiments were conducted using HBS-EP+buffer as the running buffer.

Purified nanobodies were diluted in 10 mM sodium acetate buffer (pH 5.0) and covalently immobilized onto the CM5 chip using a standard amine coupling protocol. Vaspin protein, at concentrations ranging from 0 to 100 nM, was injected over the sensor surface at a flow rate of 30 μL/min, with an association time of 30 seconds and a dissociation time of 600 seconds. Binding kinetics between Vaspin and the nanobody were analyzed using a 1:1 binding model implemented in the Biacore T200 Evaluation Software version 3.2.1.

### 1-5. Surface Plasmon Resonance (SPR) Analysis

Binding affinity was determined using a Biacore T200 system (Cytiva, Uppsala, Sweden) equipped with a CM5 sensor chip. Experiments were conducted using HBS-EP+buffer as the running buffer.

Purified nanobodies were diluted in 10 mM sodium acetate buffer (pH 5.0) and covalently immobilized onto the CM5 chip using a standard amine coupling protocol. Vaspin protein, at concentrations ranging from 0 to 100 nM, was injected over the sensor surface at a flow rate of 30 L/min, with an association time of 30 seconds and a dissociation time of 600 seconds. Binding kinetics between Vaspin and the nanobody were analyzed using a 1:1 binding model implemented in the Biacore T200 Evaluation Software version 3.2.1.

1-6. Homology Modeling and Docking Simulation of Vas-Nanobody with Vaspin

To investigate the binding mode between Vas-nanobody and Vaspin, protein-protein docking simulations were performed based on the X-ray crystal structure of Vaspin (PDB ID: 4Y3K). Since the structure of Vas-nanobody was not available, homology modeling was used to generate a 3D model.

The X-ray crystal structure of a humanized camelid single-domain antibody (PDB ID: 3EAK) was used as a template for homology modeling. Given the high sequence identity (81.7%) between the Vas-nanobody and the template, a reliable structural model was expected. The MODELLER program was employed for structural optimization using a conjugate gradient algorithm, and molecular dynamics simulations were performed to minimize steric constraints. Coordinates for loop regions were generated from randomly distorted structures connecting fixed anchor points.

The docking simulation between Vas-nanobody and Vaspin was performed using RosettaDock, a multi-scale Monte Carlo-based docking algorithm. The initial docking orientation was manually selected to position the complementarity-determining region (CDR) of the Vas-nanobody toward Arg302 of Vaspin. The Vas-nanobody was subjected to translational and rotational adjustments to optimize its orientation relative to the fixed structure of Vaspin.

Preferred binding modes were identified by rotamer packing to refine the side-chain conformations of the Vas-nanobody, followed by gradient-based rigid-body minimization. Binding energies were calculated based on van der Waals interactions, low-weighted electrostatics, implicit Gaussian solvation, directionally dependent hydrogen bonding, and rotamer-based probability functions. Among 1,000 simulated complexes, the structure with the lowest binding energy was selected as the final model of the Vaspin-Vas-nanobody complex.

### 1-7. Statistical Analysis

All data were expressed as mean±SEM. For parametric data, comparisons between two groups were performed using a two-tailed independent t-test, while comparisons among three or more groups were assessed by one-way ANOVA followed by Tukey's multiple comparison test.

For non-parametric data, comparisons between two groups were conducted using the Mann-Whitney U test or two-tailed chi-square test, and multiple group comparisons were conducted using the Kruskal-Wallis test followed by the Mann-Whitney U test. A p-value of less than 0.05 was considered statistically significant. Statistical analyses were performed using SPSS version 20 (IBM Corp., Armonk, NY, USA)

### Example 2. Effect of Vaspin Overexpression on Cartilage Degradation

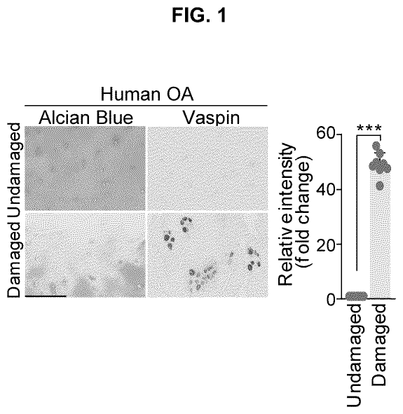

To evaluate the effect of Vaspin expression levels on cartilage degradation, the expression of Vaspin protein was assessed in intact and damaged cartilage tissues. Cartilage samples were obtained from osteoarthritis (OA) patients, and the expression level of Vaspin in intact versus damaged cartilage tissues was analyzed by Alcian blue staining and immunohistochemistry. Quantitative comparisons of expression levels were made by measuring immunostaining intensities and presenting them as graphical data. As shown in FIG. 1, Vaspin protein expression was significantly elevated in damaged cartilage compared to intact tissue.

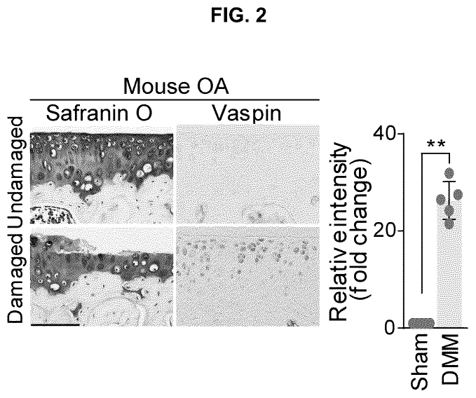

Similarly, cartilage samples were collected from a destabilization of the medial meniscus (DMM)-induced mouse model of OA. The expression of Vaspin in damaged and intact cartilage was assessed using Safranin-O staining and immunohistochemistry. Quantification of immunostaining intensities also revealed that, as shown in FIG. 2, Vaspin expression was markedly increased in the damaged cartilage of DMM-operated mice.

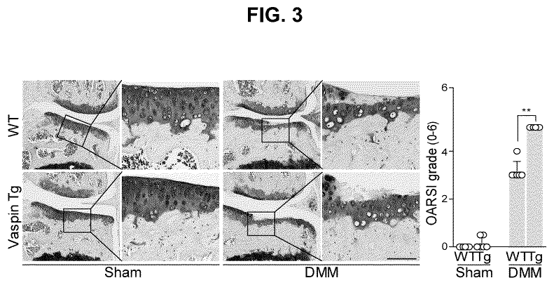

To further investigate the role of Vaspin in OA pathogenesis, a cartilage-specific Vaspin-overexpressing transgenic mouse (Col2a1-Vaspin Tg) was generated. The onset and severity of OA were evaluated in these transgenic mice under mechanical stress or upon exposure to OA-inducing stimuli. As shown in FIG. 3, DMM-operated mice exhibited higher Osteoarthritis Research Society International (OARSI) scores compared to sham-operated controls, and Col2a1-Vaspin Tg mice displayed significantly higher OARSI scores than wild-type (WT) controls. These findings indicate that cartilage-specific overexpression of Vaspin exacerbates cartilage destruction in response to DMM, suggesting a deleterious role of Vaspin in OA progression.

### Example 3. Isolation of High-Affinity Vas-Nanobody Via Bio-Panning of a VHH Phage Library

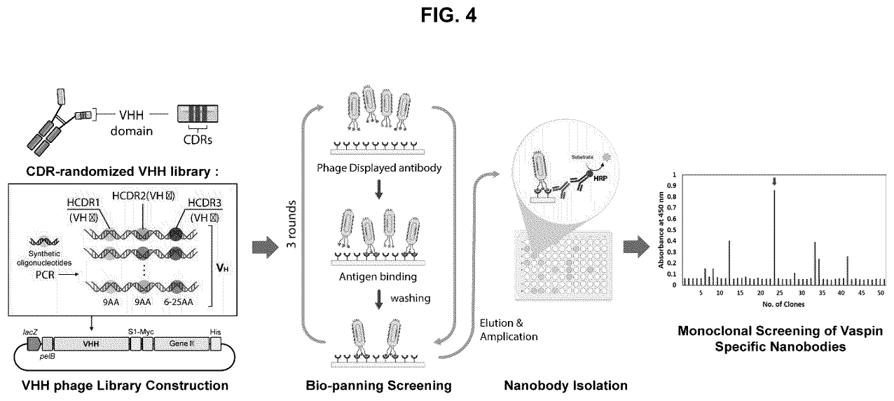

A high-affinity, Vaspin-specific monoclonal nanobody (Vas-nanobody) was isolated by bio-panning of a humanized synthetic VHH phage library. As shown in FIG. 4, bio-panning was conducted using the humanized synthetic VHH library (hNbBcll10FGLA) displaying diverse nanobody clones with variable-length complementarity-determining regions (CDRs). The library contained more than 10¹¹ unique clones with fixed lengths of 9 and 6 amino acids for CDR1 and CDR2, respectively, and variable lengths ranging from 6 to 25 amino acids for CDR3.

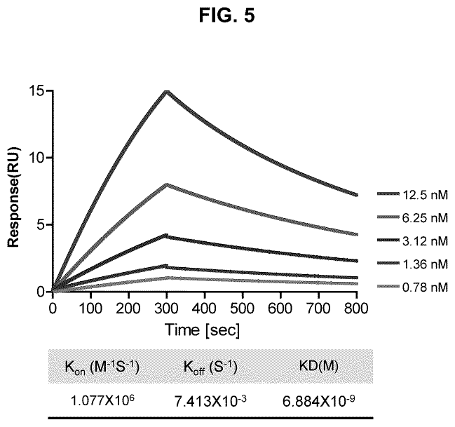

Following multiple rounds of panning, clones with strong Vaspin-binding activity were identified by ELISA and confirmed to be unique through DNA sequencing. Binding kinetics and affinity between Vaspin and the selected nanobody were measured using surface plasmon resonance (SPR). Vaspin was injected at concentrations ranging from 0.78 to 12.5 nM over a nanobody-coated low-density SPR chip. As shown in FIG. 5, the kon and koff values for the Vas-nanobody-Vaspin interaction were calculated as 1.077×10⁶ M⁻¹ s⁻¹ and 7.413×10⁻³ s⁻¹, respectively, resulting in a steady-state dissociation constant (KD) of 6.88 nM.

To investigate the structural interaction between the Vas-nanobody and Vaspin, molecular docking simulations were performed based on the X-ray crystal structure of Vaspin (PDB ID: 4Y3K). Intermolecular hydrogen bonds and van der Waals contacts were highlighted in the structural models. The docking simulation predicted the most plausible binding conformation between Vaspin and the modeled structure of the Vas-nanobody.

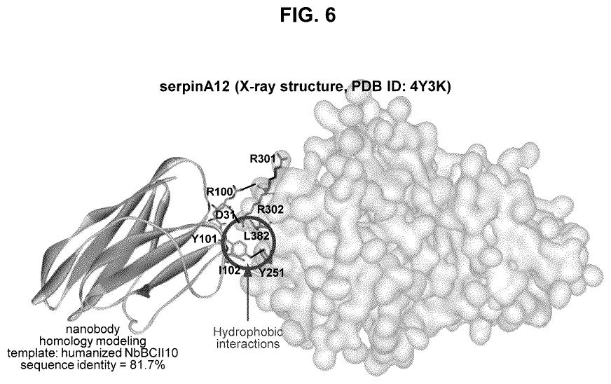

As illustrated in FIG. 6, key interactions were identified between the side chains of Arg100 and Tyr101 in the CDR region of the Vas-nanobody. Additionally, a hydrogen bond was predicted between Asp31 of the Vas-nanobody and Arg302 of Vaspin. Hydrophobic interactions were also predicted to contribute significantly to complex stabilization. Specifically, Tyr101 and Ile102 of the nanobody were predicted to interact with Leu382 and Tyr251 of Vaspin, respectively. These hydrophobic interactions are expected to shield adjacent hydrogen bonds from solvent exposure and enhance binding stability. The cooperative effects of multiple hydrophobic contacts and solvent-shielded hydrogen bonds, especially involving the charged side chains of Arg and Asp, suggest a critical role for both electrostatic and hydrophobic interactions in determining the specificity of the Vas-nanobody for Vaspin.

These findings demonstrate that the binding specificity of the Vas-nanobody to Vaspin is primarily driven by a combination of hydrogen bonding and hydrophobic interactions at the nanobody-antigen interface.

### Example 4. Isolation of High-Affinity Vas-Nanobody Via Bio-Panning of a VHH Phage Library

A high-affinity, Vaspin-specific monoclonal nanobody (Vas-nanobody) was isolated through bio-panning of a VHH phage display library. As shown in FIG. 4, the humanized synthetic VHH library (hNbBcll10FGLA) was utilized for bio-panning, in which nanobody-displaying phages were enriched against the Vaspin antigen. Following several rounds of panning, clones with strong Vaspin-binding affinities were identified by ELISA, and their uniqueness was confirmed by DNA sequencing.

The binding affinity and kinetics between Vaspin and the selected nanobody were evaluated using surface plasmon resonance (SPR). Vaspin was applied to a low-density nanobody-coated chip at concentrations ranging from 0.78 to 12.5 nM. The screened nanobody was injected into serially diluted Vaspin samples, and binding interactions were measured using SPR. As shown in FIG. 5, the association rate constant (kon) and dissociation rate constant (koff) of the Vas-nanobody-Vaspin complex were 1.077×10⁶ M⁻¹ s⁻¹ and 7.413×10⁻³ s⁻¹, respectively, yielding a dissociation constant (KD) of 6.88 nM.

The binding specificity of the Vas-nanobody for Vaspin was further assessed via dot blot analysis using nitrocellulose membranes spotted with serially diluted amounts of Vaspin protein (1000 to 31 ng). The specific interaction between the isolated nanobody and Vaspin protein was visualized using HRP-conjugated secondary antibodies. The Vas-nanobody showed a dose-dependent binding to Vaspin but no reactivity to BSA, confirming its specificity.

To further investigate the interaction between the Vas-nanobody and Vaspin, a protein-protein docking simulation was conducted based on the X-ray crystallographic structure of Vaspin (PDB ID: 4Y3K). Intermolecular hydrogen bonds were visualized as dashed lines, and van der Waals contacts were highlighted with red dotted circles. The most plausible binding conformation between the Vas-nanobody and Vaspin was predicted through computational docking.

As shown in FIG. 6, interaction was observed between the side chains of Arg100 and Tyr101 within the complementarity-determining region (CDR) of the Vas-nanobody. The computed structure of the Vaspin-Vas-nanobody complex predicted an additional hydrogen bond between Asp31 of the Vas-nanobody and Arg302 of Vaspin. The binding interaction between the Vas-nanobody and Vaspin was further enhanced by hydrophobic interactions. Specifically, Tyr101 and Ile102 in the CDR of the Vas-nanobody were predicted to interact with Leu382 and Tyr251 of Vaspin, respectively.

These hydrophobic interactions are expected to stabilize the complex by limiting access of water molecules to nearby hydrogen bonds and reinforcing intermolecular contacts. Given that these interactions are positioned near three predicted hydrogen bonds, they are likely to play a critical role in complex stabilization. The protective function of these hydrophobic contacts is further enhanced by the involvement of highly solvated ionic side chains of Arg and Asp in two adjacent hydrogen bonds.

Collectively, these results demonstrate that the specificity of the Vas-nanobody for Vaspin is attributable to a combination of multiple hydrogen bonds and hydrophobic interactions.

### Example 5. Therapeutic Efficacy of Vas-Nanobody in Osteoarthritis

The effect of the screened Vas-nanobody in suppressing osteoarthritis (OA) progression was evaluated by determining whether it could neutralize Vaspin and subsequently inhibit the Vaspin-AP-1 signaling axis in chondrocytes.

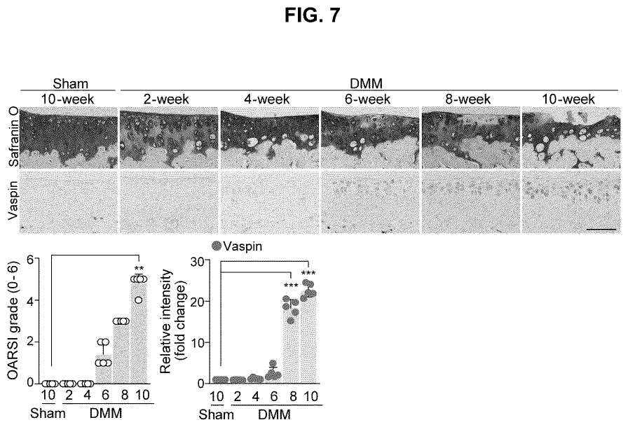

To this end, osteoarthritis was induced in mice via destabilization of the medial meniscus (DMM). Cartilage sections were collected from mice at 2, 4, 6, 8, or 10 weeks post-DMM induction. The degree of cartilage destruction was assessed via Safranin-O staining and scored using the Osteoarthritis Research Society International (OARSI) grading system. As shown in FIG. 7, progressive increases in Vaspin levels and cartilage degradation were observed beginning at week 6 post-DMM.

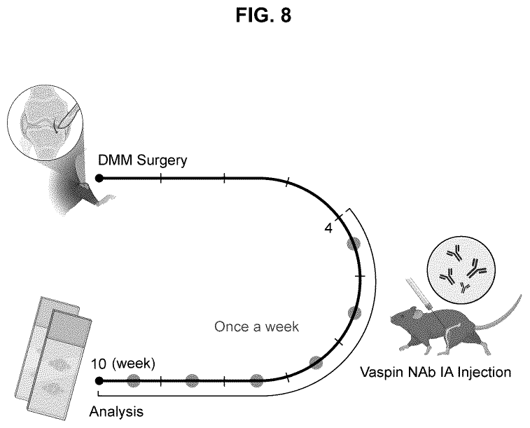

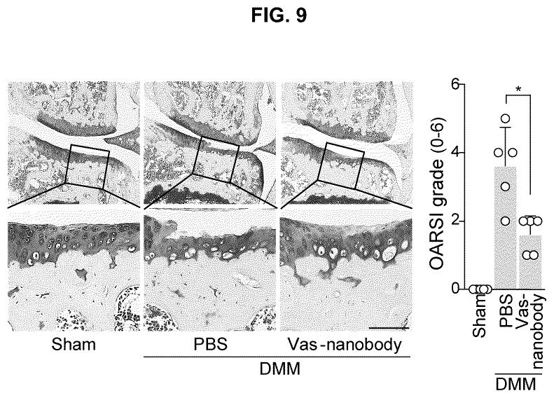

To assess the therapeutic potential of the Vas-nanobody in DMM-induced OA mice, intra-articular administration was performed. As shown in FIG. 8, mice were subjected to Sham or DMM surgery and treated weekly with either PBS (vehicle control) or Vas-nanobody (25 μg/10 μL) via intra-arterial (IA) injection for 7 consecutive weeks. At week 10, cartilage tissues were harvested, stained with Safranin-O, and scored using the OARSI grading system to evaluate cartilage damage. As shown in FIG. 9, repeated IA injections of Vas-nanobody markedly reduced DMM-induced cartilage destruction.

These results collectively demonstrate that the Vas-nanobody effectively captures Vaspin and inhibits the Vaspin-AP-1 signaling axis, thereby preventing or reducing osteoarthritis progression.

While specific embodiments of the present invention have been described in detail, it is to be understood that the disclosed embodiments are merely illustrative and not limiting. The scope of the invention should be construed in accordance with the appended claims and their equivalents.

All numerical ranges provided herein include the stated values. The upper and lower limits of any numeric range may be independently included or excluded. Additionally, all ranges encompass narrower subranges within the broader limits specified.

1. A nanobody or an antigen-binding fragment thereof that specifically binds to vaspin, wherein the nanobody comprises a CDR1 having the amino acid sequence set forth in SEQ ID NO: 2, a CDR2 having the amino acid sequence set forth in SEQ ID NO: 3, and a CDR3 having the amino acid sequence set forth in SEQ ID NO: 4.

2. The nanobody or antigen-binding fragment thereof according to claim 1, wherein the nanobody is a humanized recombinant nanobody.

3. The nanobody or antigen-binding fragment thereof according to claim 1, wherein the nanobody comprises an arginine (Arg, R) residue at position 100.

4. The nanobody or antigen-binding fragment thereof according to claim 1, wherein the nanobody comprises a tyrosine (Tyr, Y) residue at position 101.

5. The nanobody or antigen-binding fragment thereof according to claim 1, wherein the nanobody comprises an aspartic acid (Asp, D) residue at position 31.

6. The nanobody or antigen-binding fragment thereof according to claim 1, wherein the nanobody comprises an isoleucine (Ile, I) residue at position 102.

7. The nanobody or antigen-binding fragment thereof according to claim 1, wherein the nanobody comprises the amino acid sequence set forth in SEQ ID NO: 5.

8. A polynucleotide encoding the nanobody or antigen-binding fragment thereof according to claim 1.

9. A pharmaceutical composition for the prevention or treatment of osteoarthritis, comprising the nanobody or antigen-binding fragment thereof according to claim 1.
